(19) United States
(12) Patent Application Publication
(10) Pub. No.: US 2011/0040165 A1
(43) Pub. Date: Feb. 17, 2011

(54) INTEGRATED CARTRIDGE AND TUB ASSEMBLY

(76) Inventor: George M. Williams, III, Dublin, CA (US)

Correspondence Address: Abbott Diabetes Care Inc.;Bozicevic, Field & Francis LLP, 1900 University Ave, Suite 200, East Palo Alto, CA, 94303, US

(21) Appl. No.: 12/541,068

(22) Filed: Aug. 13, 2009

(51) Int. Cl. A61B 5/00; B65D 83/00

(52) U.S. Cl. 600/365; 206/305

(57) ABSTRACT

An assembly for storing and dispensing a plurality of integrated lancet and test strips for use in a testing meter is provided. The assembly comprises a cartridge portion for housing a plurality of integrated lancet and test strips, and a tub portion coupled to the cartridge portion and providing a surface for advancing a single integrated lancet and test strip for use in the testing meter. A system is also provided which includes the assembly and a meter for determining an analyte concentration.

### BACKGROUND OF THE INVENTION

The prevalence of diabetes is increasing markedly in the world. At this time, diagnosed diabetics represent about 3% of the population of the United States. It is believed that the actual number of diabetics in the United States is much higher. Diabetes can lead to numerous complications, such as, for example, retinopathy, nephropathy, and neuropathy.

The most important factor for reducing diabetes-associated complications is the maintenance of an appropriate level of glucose in the blood stream.

The maintenance of the appropriate level of glucose in the blood stream may prevent and even reverse some of the effects of diabetes.

Analyte, e.g., glucose, monitoring devices known in the art have operated on the principle of taking blood from an individual by a variety of methods, such as by means of a needle or a lancet. The individual places a paper strip carrying reagents with the blood into a blood glucose meter and then applies a blood sample to the paper strip for measurement of glucose concentration by optical or electrochemical techniques.

Previously, medical devices for monitoring the level of glucose in the blood stream have required that an individual have separately available a needle or a lancet for extracting blood from the individual, test strips carrying reagents for bringing about a chemical reaction with the glucose in the blood stream and generating an optical or electrochemical signal, and a blood glucose, meter for reading the results of the reaction, thereby indicating the level of glucose in the blood stream. The level of glucose, when measured by a glucose, meter, is read from the strip by an optical or electrochemical meter.

Medical diagnostic devices have started to use lancet and test strips that are integrated. A magazine of integrated lancet and test strips are manufactured in cartridges for insertion within the medical diagnostic device. The cartridges are open at the dispensing end of the cartridge and expose the integrated lancet and test strips to environment elements and potential contaminants—e.g., debris, dirt, liquids, oil, etc. Contaminants may also exist within the meter where the open cartridge mates with the testing meter. These contaminants may accumulate at the mating site and may eventually compromise the integrated lancet and test strip when the cartridge is inserted into the meter.

It is important that stored integrated lancet and strips be protected against contaminants such as ambient humidity, dirt, debris, liquids, oils, etc. Humidity will degrade the chemicals on the strip, rendering it unusable. Moreover, the use of a compromised strip may provide an inaccurate test result.

### SUMMARY OF THE INVENTION

An assembly for storing and advancing a plurality of integrated lancet and test strips for use in a testing meter, and a system including the assembly and testing meter, are provided. The assembly comprises a cartridge portion for housing a plurality of integrated lancet and test strips, and a tub portion coupled to the cartridge portion and providing a surface for advancing a single integrated lancet and test strip for use in the testing meter.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A illustrates a perspective view of an integrated cartridge and tub assembly, according to certain embodiments.

FIG. 1B illustrates a cross-sectional view of an integrated cartridge and tub assembly, according to certain embodiments.

FIG. 1C illustrates an exploded perspective view of an integrated cartridge and tub assembly, according to certain embodiments.

FIGS. 2A-B illustrate a perspective view of an integrated cartridge and tub assembly and corresponding mating portion of a testing meter, according to certain embodiments.

FIG. 2C illustrates a planar view of an integrated cartridge and tub assembly, according to certain embodiments.

FIGS. 3A-B illustrate a cross-sectional views of a tub portion of an assembly when engaging with a testing meter, according to certain embodiments.

FIG. 4 illustrates a cross-sectional view of an integrated cartridge and tub assembly, according to certain embodiments.

FIG. 5 illustrates a cross-sectional view of an integrated cartridge and tub assembly, according to certain embodiments.

FIGS. 6A-B illustrate an opening formed in an integrated cartridge and tub assembly upon application of a force by a testing meter, according to certain embodiments.

FIG. 7A illustrates openings formed in the tub portion of an assembly, according to certain embodiments.

FIG. 7B illustrates openings formed in the cartridge portion of an assembly, according to certain embodiments.

FIGS. 8A-B illustrate openings formed in an integrated cartridge and tub assembly upon application of a force by a testing meter, according to certain embodiments.

FIGS. 9A-B illustrate openings formed in an ICTA upon application of a force by a testing meter, according to certain embodiments.

FIGS. 10A-P illustrates an operational sequence of a system including a meter and integrated cartridge and tub assembly, according to certain embodiments.

FIGS. 11A-B illustrate a planar view of an integrated cartridge and tub assembly, according to certain embodiments.

### DETAILED DESCRIPTION OF THE INVENTION

Before the present inventions are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

An integrated cartridge and tub assembly (ICTA) for storing and advancing a plurality of integrated lancet and test strips for use in a testing meter is provided. The assembly comprises a cartridge portion for housing the plurality of integrated lancet and test strips, a tub portion having a surface for advancing a single integrated lancet and test strip for use in the meter. Furthermore, a resilient biasing element may be coupled to the tub portion and cartridge portion and apply closing forces to the tub portion and the cartridge portion so that the tub portion is positioned in a relative first position from the cartridge portion.

A sealing element may be positioned between the cartridge portion and the tub portion to provide an air-tight and moisture tight environment (e.g., from ambient humidity and other contaminants) for the stored plurality of integrated lancet and test strips. It should be understood that sealing elements (also referred to herein as “seals”) are referred to herein as providing “an air-tight and moisture-tight seal”; or providing for “an air-tight and moisture-tight environment” or “sealed environment” for the stored plurality of integrated lancet and test strips. It should be understood that sealing elements are typically made from a substantially air-impermeable, moisture-impermeable material, such as, for example, rubber, elastomeric, or a polymeric material; and, for all intents and purposes herein, are referred to as providing “an air-tight and moisture-tight” seal or environment (or, a “sealed environment”). The resilient biasing element applies the closing forces to the tub portion and the cartridge portion necessary to create the air-tight and moisture-tight (e.g., against humidity) seal between the two components.

The sealed environment protects the integrated lancet and test strip from environmental elements which may compromise the integrated lancet and test strip—e.g., ambient humidity, debris, dirt, liquids, oil, etc. As stated before, humidity may degrade the chemicals on the strip to the point of making it unusable. And further, a compromised strip may provide an inaccurate test result.

When the ICTA is inserted into a testing meter, the ICTA engages with an engagement element in the testing meter. The engagement element may be, for example, a wall in which the tub portion abuts against, a rotary lever arm, any variety of male/female connectors, etc. Moreover, the engagement element may engage tub portion in a variety of ways (e.g., latch, hook, abut, snap, etc.). Further, the engagement element may simply hold the ICTA in place, or may apply a force to the ICTA (e.g., by applying the force to tub portion 110). Depending on the type of engagement mechanism implemented between the tub portion and the engagement element, the engagement element may apply a displacing force to ICTA by pushing the engagement element against the tub portion, or pulling on the tub portion with the engagement element.

When a displacing force is applied to the assembly, the resilient biasing element is stressed (e.g., stretched or compressed) and the tub portion is relatively displaced to a relative second position from the cartridge portion, wherein an opening is formed in the assembly when the tub portion is in the relative second position. When the displacing force is removed the tub portion returns to the relative first position from the cartridge portion under closing forces of the resilient biasing element, thus closing the opening and reestablishing a sealed environment (i.e., an air-tight and moisture-tight environment). In certain embodiments, a sealing element is not used and a sealed environment is not provided.

In some embodiments, the ICTA is a disposable such that the user is provided with a fresh seal for every ICTA used, and contamination issues are further ensured. In other embodiments, the cartridge portion 105 and tub portion 110 are removably coupled to allow for replaceable sets of plurality of integrated lancet and test strips.

A system for analyte monitoring is also provided. The system comprises a meter for determining an analyte concentration, and an ICTA for storing and advancing a plurality of integrated lancet and test strips for use by the meter during a determination.

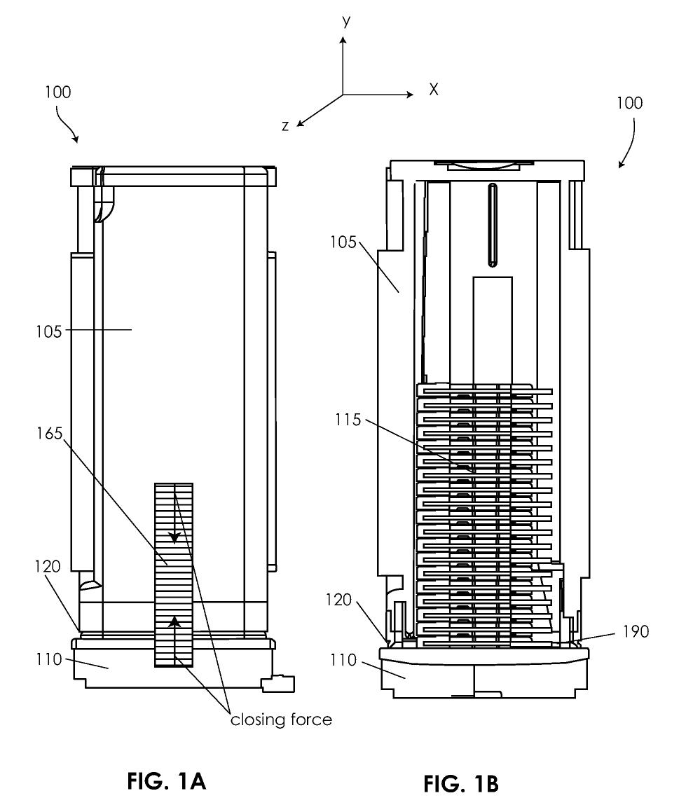

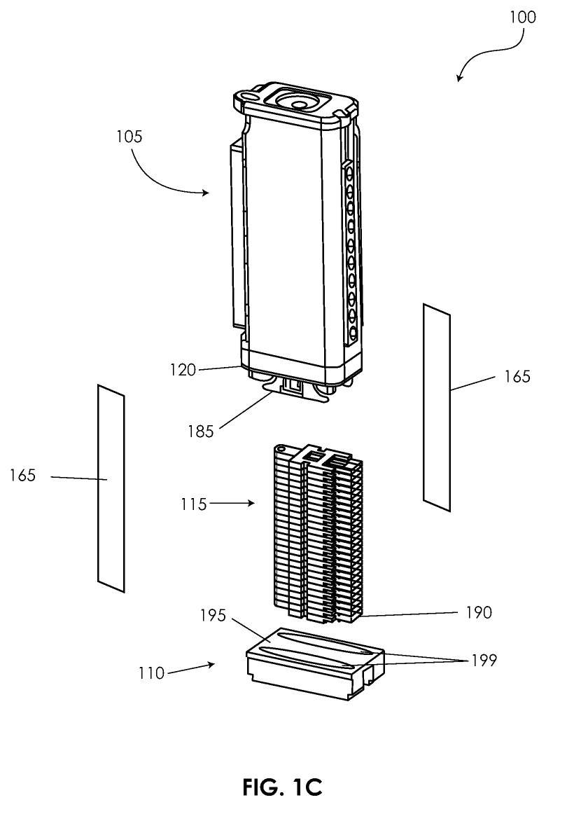

FIGS. 1-10 and their descriptions are provided to better understand the underlying principles of a novel apparatus, system and method which overcomes the contamination issues presented.

The cartridge portion of the ICTA provides a housing for storing a plurality of integrated lancet and test strips. The cartridge portion may include inserts which hold and/or align the plurality of integrated lancet and test strips for feeding to the testing meter. A tub portion provides a surface for which a single integrated lancet and test strip is advanced into the meter. A surface of the tub portion may include wells in which inserts from the cartridge portion may extend into. An advancing mechanism advances the next integrated lancet and test strip along surface and out of the ICTA for use in the testing meter. An example of advancing mechanism, such as a pusher and chain system are described in U.S. patent application Ser. Nos. 11/535,985, 11/535,986, 12/488,181, entireties which are hereby incorporated by reference.

FIG. 1A illustrates a front planar view of an ICTA, according to certain embodiments. ICTA 100 comprises a cartridge portion 105, tub portion 110, and resilient biasing element 165. Resilient biasing element 165 couples the cartridge portion to the tub portion and applies closing forces to the cartridge portion 105 and the tub portion 110 such that the two are pressed towards each other. The closing force applied to the tub portion 110 and the closing force applied to the cartridge portion 105 are in opposite directions such that the cartridge portion 105 and the tub portion 110 are held together, positioning the tub portion 110 in a relative first position from the cartridge portion 105. In the relative first position, the cartridge portion 105 and tub portion 110 provide a closed environment for housing the integrated lancet and test strips (not shown in FIG. 1A). Sealing element 120 is shown positioned between the tub portion 110 and cartridge portion 105 and provides an air-tight and moisture-tight seal. It should be understood that while only one resilient biasing element is shown, more than one resilient biasing element may be used. Any type of resilient biasing element may be used, as long as the closing forces applied by the resilient biasing element are in directions to hold the cartridge portion 105 and tub portion 110 together in the relative first position. For example, a resilient biasing element may be any elastic component that returns to its original form when stressed (e.g., stretched and/or compressed)—e.g., an elastic band, spring, etc. Thus, the resilient biasing element may be stressed when coupled to the tub portion 110 and cartridge portion 105, thus applying closing forces to the tub portion 110 and cartridge portion 105.

For example, resilient biasing element 165 in FIG. 1A pulls the cartridge portion 105 in the negative y-direction and the tub portion 110 in the positive y-direction. Furthermore, the resilient biasing element may be located in a variety of places—e.g., on the inside and/or outside of the ICTA (i.e., on the inside and/or outside of cartridge portion 105 and tub portion 110. In certain alternative embodiments, seal 120 is not present and an air-tight and moisture-tight environment is not provided for.

When a displacing force is applied to the ICTA by the testing meter (e.g., engagement element), the resilient biasing elements is stressed even further to allow the tub portion 110 to be relatively displaced to a relative second position from the cartridge portion 105 (discussed in more detail later). When the tub portion 110 is at the relative second position, the next integrated lancet and test strip 190 may be advanced out an opening in the ICTA. When the displacing force is removed, the resilient biasing element returns tub portion 110 to the relative first position from cartridge portion 105.

FIG. 1B illustrates a cross-sectional view of the ICTA shown in FIG. 1A, according to certain embodiments. As shown, tub portion 110 is in a relative first position from cartridge portion 105, with a plurality of integrated lancet and test strips 115 stored within ICTA 100. Sealing element 120 between the tub portion 110 and cartridge portion 105 provides an air-tight and moisture-tight seal between the two. Thus, an air-tight and moisture-tight environment is provided for the plurality of integrated lancet and test strips 115.

FIG. 1C illustrates an exploded view of an ICTA, according to certain embodiments. ICTA 100 comprises cartridge portion 105 and tub portion 110. Resilient biasing element 165 couples the cartridge portion 105 to the tub portion 110 and applies closing forces to the cartridge portion 105 and the tub portion.

In certain alternative embodiments, the cartridge portion 105 and tub portion 110 are coupled together by other mechanisms than resilient biasing elements, such as for example, a snap-type retention mechanism that snaps the two together. In such embodiments, a sealing element 120 may be used to provide an air-tight and moisture-tight seal between the two.

When the displacing force is applied by the testing meter, the tub portion 110 and cartridge portion 105 are snapped apart, allowing allow the tub portion 110 to be relatively displaced to a relative second position from the cartridge portion 105. When the tub portion is at the relative second position, the next integrated lancet and test strip 190 may be advanced out an opening in the ICTA. Subsequently, the engagement element 220 may return the tub portion 110 to the first relative position from the cartridge portion 105, thus snapping them together again to form an air-tight and moisture tight seal again.

In some embodiments, the cartridge portion 105 and tub portion 110 are irremovably coupled such that replaceable sets of plurality of integrated lancet and test strips cannot be inserted into the ICTA. In such embodiments, the ICTA is disposable and the user is provided with a fresh seal for every ICTA. In certain alternative embodiments, the cartridge portion 105 and tub portion 110 are removably coupled to allow for replaceable sets of plurality of integrated lancet and test strips 115. However, whether removably coupled or irremovably coupled, the ICTA (including both the cartridge portion and tub portion) is inserted into the testing meter for use.

With the tub portion coupled to the cartridge portion, the integrated lancet and test strips are provided with protection from damage, contamination, etc. Furthermore, the ICTA may comprise sealing elements (also referred to herein as “seals”) to provide an air-tight and moisture-tight environment for the stored plurality of integrated lancet and test strips. It should be understood that sealing elements are typically made from a substantially air-impermeable, moisture-impermeable material, such as, for example, rubber, elastomeric, or a polymeric material; and, for all intents and purposes herein, are referred to as providing “an air-tight and moisture-tight” seal or environment (or in other words, a “sealed environment”).

As stated earlier, when the ICTA is inserted into a testing meter, the ICTA engages with an engagement element in the testing meter. The engagement element may be, for example, a wall in which the tub portion 110 abuts against, a rotary lever arm, any variety of male/female connectors, etc. Moreover, the engagement element may engage tub portion 110 in a variety of ways (e.g., latch, hook, abut, snap, etc.). The engagement element may simply hold the ICTA in place, or may apply a displacing force to the ICTA (e.g., by applying a displacing force to tub portion 110) to relatively displace tub portion 110 to a relative second position from cartridge portion 105.

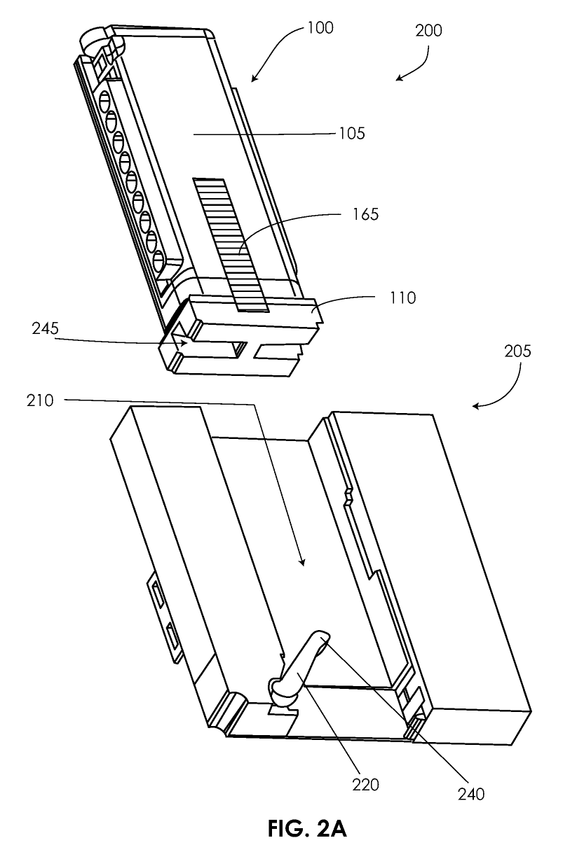

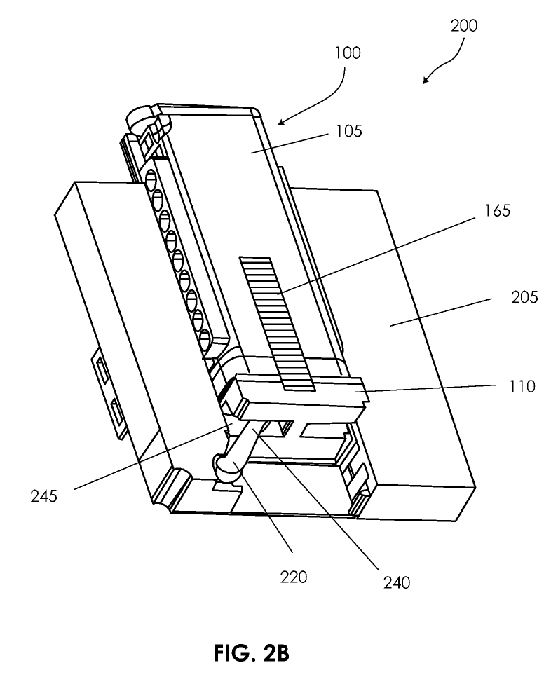

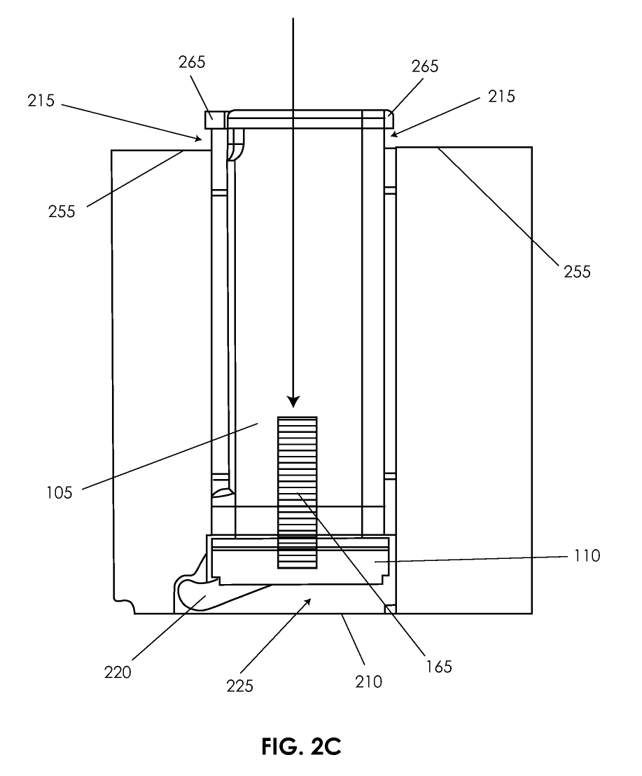

For example, FIGS. 2A-C illustrate an engagement mechanism implemented in system 200 comprising ICTA 100 and meter 205, according to certain embodiments. It should be understood that that only a portion of meter 205 is shown-the portion that mates with the ICTA 100. As shown in FIG. 2A, ICTA 100 comprises cartridge portion 105 and tub portion 110 and resilient biasing element 156. ICTA 100 is inserted within cavity 210 of meter 205. As shown in FIG. 2B, when ICTA 100 is inserted into cavity 210, tub portion 110 engages with engagement element 220 of meter 205. Engagement element 220 is shown as a rotary lever arm comprising a distal end 240 which fits within tub

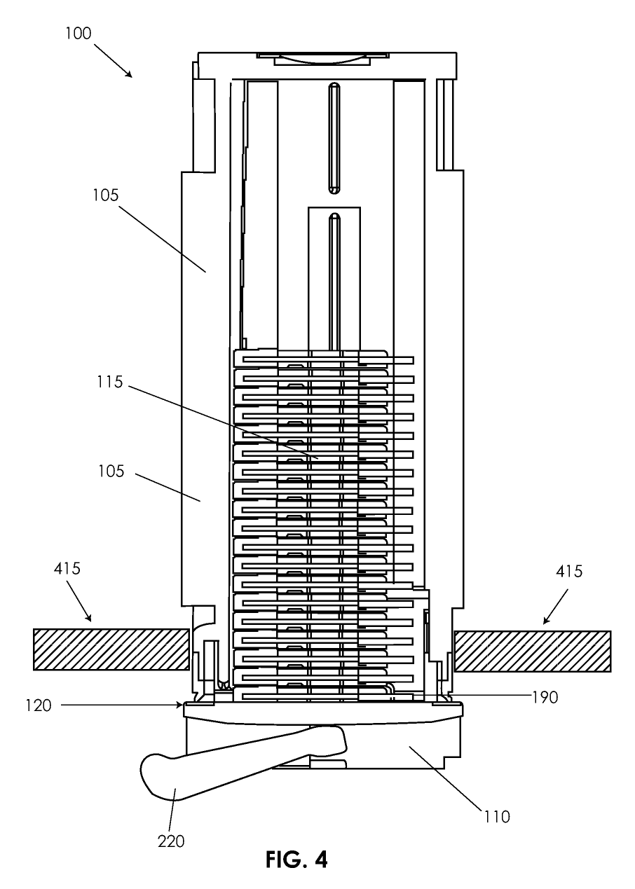

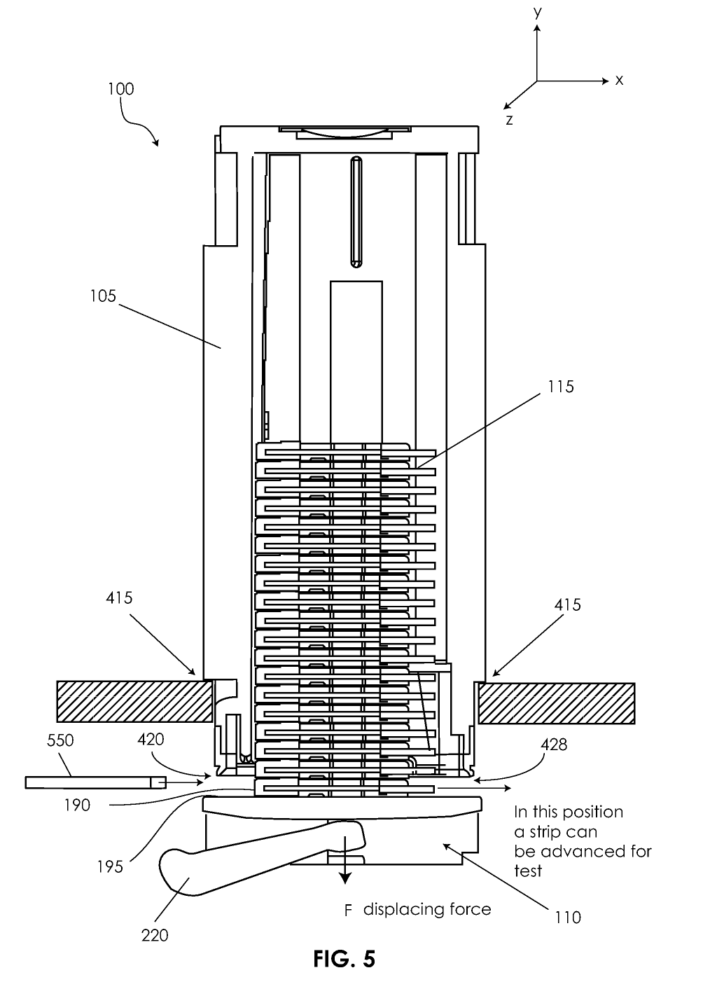

When ICTA 100 is completely inserted into meter 205, stops may be implemented to stop ICTA 100 at a certain point, and space may be provided to allow engagement element 220 to relatively displace tub portion 110 to a relative second position from cartridge portion 105. For example, FIG. 2C illustrates a planar view of ICTA 100 inserted into cavity 210 of meter 205, according to certain embodiments. When ICTA 100 is completely inserted into meter 205, stops 215 stop ICTA 100 from entering past a certain point. Stops 215 are shown in FIG. 2C as a surface 255 of the meter's chassis that contacts a protruding lip 265 of the cartridge housing body to stop the cartridge when fully inserted. Note that in FIG. 2C, the surface 215 has not yet contacted the protruding lip 265. Space 225 is provided between tub portion 110 and a bottom surface of cavity 210. Because of space 225 and stops 215, engagement element 220 may pull tub portion 110 away from cartridge portion 105 to a relative second position. It should be understood that other stopping mechanisms may be implemented to stop ICTA 100 when fully inserted into meter 205 and allowing engagement element 220 to relatively displace the tub portion 110 to a relative second position from the cartridge portion 105. For example, FIGS. 4-5 shows stops 415 at a location lower in the cavity of the meter.

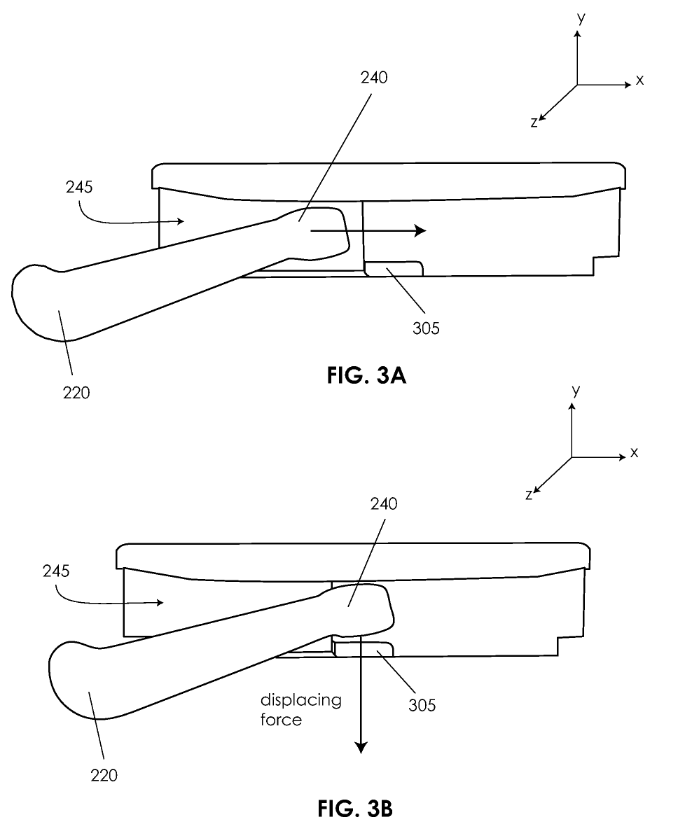

FIGS. 3A-B illustrate a close up view of engagement element 220 engaging tub portion 110, according to certain embodiments. Tub portion 110 includes barrier 305 over the central portion of tub groove 245. As shown in FIG. 3A, as ICTA 100 is inserted into meter 205, distal end 240 of engagement element 220 slides towards the center of tub groove 245 until distal end 240 is above barrier 305 (as shown in FIG. 3B). With distal end 240 above barrier 305, engagement element 220 may now apply a displacing force to barrier portion 305 to relatively displace tub portion 110 to a relative second position from cartridge portion 105. FIGS. 3A-B illustrate a displacing force in the negative y-direction. In certain alternative embodiments, a displacing force is applied in the positive y-direction.

Because cartridge portion 105 and tub portion 110 are coupled together to store a plurality of integrated lancet and test strips, one or more openings in the ICTA are needed to allow an integrated lancet and test strip to advance out of the ICTA for use by the testing meter.

An opening within the ICTA is formed after the ICTA is inserted into the testing meter and a displacing force applied to the ICTA by the engagement element of the testing meter. As shown above, the ICTA may comprise one or more resilient biasing elements coupled to the cartridge portion and tub portion in order to position the tub portion in a relative first position from the cartridge portion. When a displacing force is applied to the ICTA by the testing meter (e.g., engagement element), the resilient biasing element allows the tub portion to be relatively displaced to a relative second position from the cartridge portion. When the tub portion is at the relative second position, the next integrated lancet and test strip may be advanced out an opening in the ICTA. It should be understood that either the tub portion or cartridge portion, or both tub portion and cartridge portion, may be displaced to relatively displace the tub portion to the relative second position from the cartridge portion.

FIGS. 4-5 illustrate openings formed in an ICTA upon application of a displacing force by a testing meter, according to certain embodiments. As shown, ICTA 100 comprises cartridge portion 105 coupled to tub portion 110. Tub portion 110 is engaged with engagement element 220 of meter 205. Resilient biasing element (not shown in the cross sectional view in FIGS. 4-5) is coupled to tub portion 110 and cartridge portion 105 and provides closing forces to position tub portion 110 in the relative first position from cartridge 105.

FIG. 4 shows tub portion 110 in a relative first position from cartridge portion 105. In the relative first position, tub portion 105 and cartridge portion 105 are held together by resilient biasing element. Sealing element 120 is shown positioned between the tub portion 110 and cartridge portion 105 to provide an air-tight and moisture-tight environment for the plurality of integrated lancet and test strips 11 5. The next integrated lancet and test strip 190 to be advanced is located on or near surface 195 of tub portion 110. In certain embodiments, cartridge portion 105 includes inserts 185 which hold and/or align the plurality of integrated lancet and test strips within the cartridge portion 105. In certain embodiments, inserts 185 extend into tub wells 199 and allow the next integrated lancet and test strip 190 to rest on the surface 195 of tub portion 110.

As shown in FIG. 5, engagement element applies a displacing force F to ICTA 100 and moves ICTA 100 downward (i.e., in the negative y-direction) so that stops 415 contact the cartridge portion 105. As engagement element continues to apply the displacing force, tub portion 110 is relatively 420, 421 are formed in ICTA 100. Resilient biasing element 165 (not shown in FIGS. 4-5) is stressed as tub portion 110 is relatively displaced to the relative second position from cartridge portion 105._A distance of the relative displacing of the tub portion 110 is equal to at least one thickness of an integrated lancet and test strip. An advancing mechanism (e.g., pusher and chain system) 550, for example, may enter opening 420 and push next integrated lancet and test strip 190 along surface 195 and out opening 420 for use within testing meter 205. When the example pusher and chain system 550 is retracted, engagement element 220 returns tub portion 110 to its relative first position from cartridge 105 as shown in FIG. 4, providing once again an air-tight and moisture-tight seal to protect the plurality of integrated lancet and test strips 115. It should be understood that openings 420, 421 are referred to as two separate openings to facilitate understanding, and that one or more openings may actually be formed depending on the specific construction design of the ICTA.

In certain alternative embodiments, stops 415 contact cartridge portion 105 when fully inserted within testing meter 205 and engaged with engagement element 220. When engagement element 220 applies a displacing force to ICTA, stops 415 are already contacting cartridge portion 105 and tub portion 110 is relatively displaced to the relative second position from cartridge portion 105.

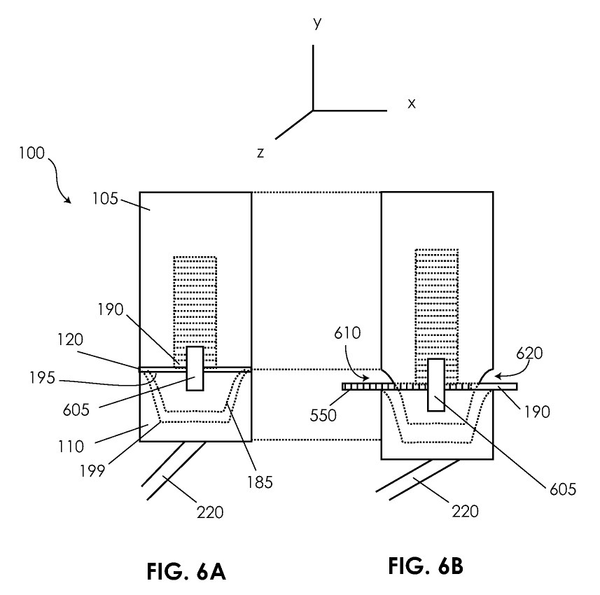

At stated above, in certain embodiments, cartridge portion 105 includes inserts 185 which hold and/or align the plurality of integrated lancet and test strips within the cartridge portion 105. As shown in FIGS. 6A-B, in certain embodiments, inserts 185 extend into tub wells 199 and allow the next integrated lancet and test strip 190 to rest on the surface 195 of tub portion 110. FIGS. 6A-B illustrate resilient biasing elements providing for openings in an ICTA upon application of a force by a testing meter, according to certain embodiments. As shown, ICTA 100 comprises cartridge portion 105 coupled to tub portion 110. Tub portion 110 is engaged with engagement element 220 of meter 205. Resilient biasing element 165 is shown coupled to tub portion 110 and to cartridge portion 105 in a manner so that closing forces are provided to maintain tub portion 110 against cartridge portion 105. In other words, resilient biasing element 165 pulls the cartridge portion 105 in the negative y-direction and tub portion 110 in the positive y-direction.

FIG. 6A shows tub portion 110 in a relative first position from cartridge portion 105, as maintained by resilient biasing element 165. Sealing element 120 is shown between cartridge portion 105 and tub portion 110 to provide an air-tight and moisture-tight environment for the plurality of integrated lancet and test strips 115. The next integrated lancet and test strip 190 to be advanced is located on or near surface 195 of tub portion 110. As shown, cartridge portion 105 includes inserts 185 which hold and/or align the plurality of integrated lancet and test strips within the cartridge portion 105. Inserts 185 extend into tub wells 199 and allow the next integrated lancet and test strip 190 to rest on the surface 195 of tub portion 110.

FIG. 6B illustrates tub portion 110 in a relative second position from cartridge portion 105 after engagement element 220 applies a displacing force F to ICTA 100. Engagement element 220 pulls (i.e., in the negative y-direction) tub portion 110 away from cartridge 105 (e.g., as shown in FIGS. 3A-B) to relatively displace tub portion 110 to a relative second position from cartridge portion 105. Resilient biasing element 165 is stressed as engagement element 220 applies a displacing force F to tub portion 110. A distance of the relative displacing of the tub portion 110 is equal to at least one thickness of an integrated lancet and test strip. When tub portion 110 is in the relative second position, inserts 185 are not extended as far into wells 199 as when in the relative first position. Also, openings 610, 620 are formed between the tub portion 110 and the cartridge portion 105. An advancing mechanism (e.g., pusher and chain system) 550, for example, may enter opening 610 and push next integrated lancet and test strip 190 along surface 195 and out opening 620 for use within testing meter 205. When the example pusher and chain system 550 is retracted, engagement element 220 returns tub portion 110 to its relative first position from cartridge 105 under the closing force applied by resilient biasing element 165.

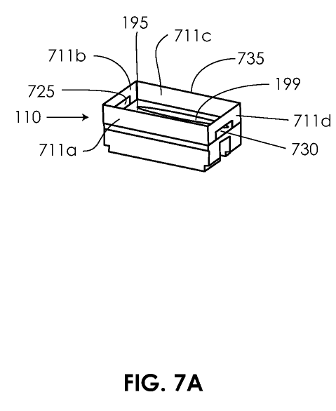

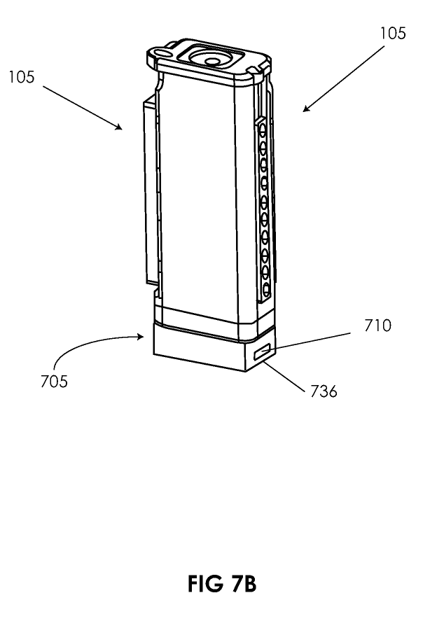

FIGS. 7A-B, 8A-B, 9A-B, and 11A-B provide additional alternative embodiments for ICTA 100 where an opening in the ICTA are manufactured in the ICTA itself and either left open or obstructed with or without a seal. For example, the cartridge portion or tub portion may include an opening in which an integrated lancet and test strip may be advanced out of for use in the testing meter. FIG. 7A illustrates a perspective view of openings formed in the tub portion of an ICTA, according to certain embodiments. As shown, tub portion 110 comprises walls 711a,711b,711c,711d, with walls 711b,711d of tub portion 710 including openings 725,730. Top edge 735 of walls 711a, 711b, 711c, 711d of tub portion 110 is coupled to cartridge portion 105. A surface 195 of the tub portion 110 may include wells 199 in which inserts 185 from the cartridge portion 105 may extend into. An advancing mechanism 550 advances the next integrated lancet and test strip 190 along surface 195 and out of the ICTA 100 for use in the testing meter 205.

FIG. 7B illustrates a perspective view of ICTA 100 wherein the cartridge portion 105 has openings 705,710 formed within it. Bottom edge 736 of cartridge portion 105 couples to tub portion 110.

In FIGS. 7A and 7B, an integrated lancet and test strip from the plurality may be advanced out one of the openings for use within the testing meter. For instance, an advancing mechanism (e.g., pusher and chain system) (not shown) from meter 205 may enter an opening (opening 725 in FIG. 7A and opening 705 in FIG. 7B) and push an integrated lancet and test strip out an opening (opening 730 in FIG. 7A and opening 710 in FIG. 7B) for use within meter 205. Since when engagement element 220 engages tub portion 110, openings are already present, no further displacing force is required.

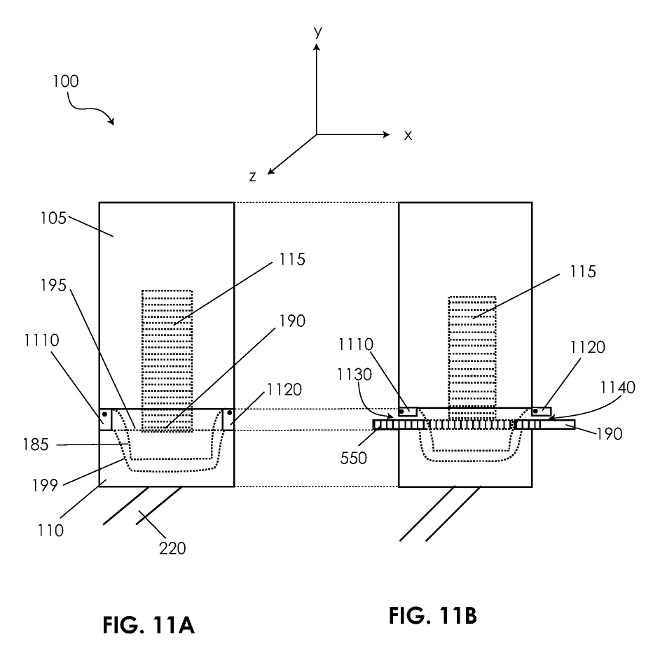

In some alternative embodiments, obstructed opening may become unobstructed to form an opening after the ICTA is inserted in the testing meter. For instance, obstructing elements may obstruct an opening in the ICTA until an integrated lancet and test strip is to be advanced out of the ICTA for use within the meter. Obstructing elements may include any variety of mechanisms, for example, flaps, doors, etc. FIGS. 11A-B illustrate obstruction elements used to form an opening in an ICTA, according to certain embodiments. ICTA 100 comprises obstruction elements 1110,1120 in the form of flaps which, when flapped open, form openings 1130,1140, respectively, as shown in FIG. 11B. And, as shown in FIG. 11A, when flapped closed, obstruction elements 1110,1120 obstruct openings 1130,1140, respectively, such that ICTA 100 does not have any openings.

The obstruction elements become unobstructed in a variety of ways. For example, an advancing mechanism (e.g., pusher and chain system) 550 may cause the obstructing elements such as flaps to become unobstructed (i.e., flapped open). Alternatively, the advancing mechanism may not cause the obstruction elements to become unobstructed. For example, a spring and hinge mechanism may be implemented so that the obstruction elements (e.g., hinged doors) swing open when a force is applied to the ICTA by the testing meter.

In some embodiments, obstruction elements 1110,1120 form a seal with ICTA 100 to form a sealed environment for the plurality of integrated lancet and test strips 115. When obstruction elements 1110,1120 are flapped open to form openings 1130,1140, respectively, as shown in FIG. 11B, an advancing mechanism (e.g., pusher and chain system) 550, for example, may enter opening 1130 and push an integrated lancet and test strip out opening 1140 along surface 195 of tub portion 110 for use within testing meter 205. When the pusher and chain system 550 are retracted, obstruction elements 1110,1120 are flapped closed again to maintain the sealed environment. In some alternative embodiments, obstruction elements 1110,1120 are present in the cartridge portion of ICTA 100 rather than the tub portion.

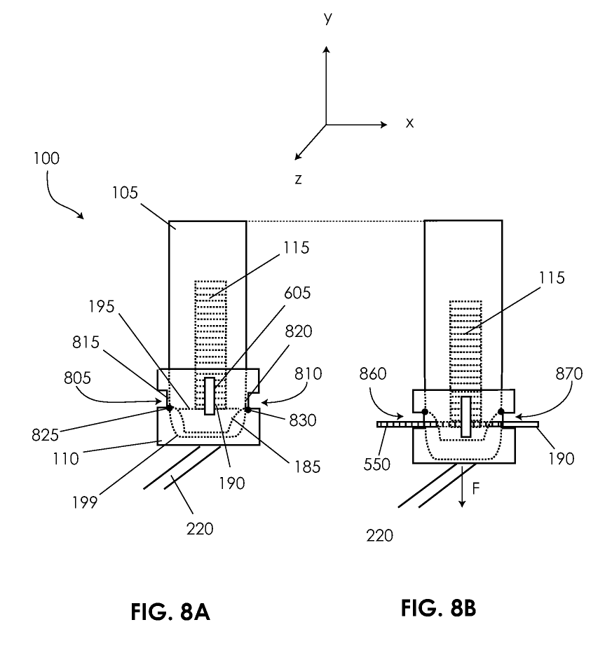

FIGS. 8A-B illustrate openings formed in an ICTA upon application of a force by a testing meter, according to certain embodiments. As shown, ICTA 100 comprises cartridge portion 105 coupled to tub portion 110. Tub portion 110 is engaged with engagement element 220 of meter 205. Resilient biasing element 165 is coupled to tub portion 110 and to cartridge portion 105, and pushes the cartridge portion 105 in the negative y-direction and tub portion 110 in the positive y-direction.

FIG. 8A shows tub portion 110 in a relative first position from cartridge portion 105, as maintained by resilient biasing element 165. In the relative first position, tub portion 110 comprises obstructed openings 805,810, which are obstructed by surfaces 815,820 of cartridge portion 105. Surfaces 815,820 are acting as obstructing elements. Optional seals 825,830 may be included to provide for an air-tight and moisture tight environment for the plurality of integrated lancet and test strips when the tub portion 110 is in the relative first position from the cartridge portion 105. The next integrated lancet and test strip 190 to be advanced is located on surface 195 of tub portion 110. Inserts 185 of cartridge portion 105 is shown extending into tub wells 199.

FIG. 8B illustrates tub portion 110 in a relative second position from cartridge portion 105 after engagement element 220 applies a displacing force F to ICTA 100 (e.g., in the negative y-direction). Engagement element 220 pulls tub portion 110 away from cartridge 105 to relatively displace tub portion 110 from cartridge portion 105. Resilient biasing element 165 is stressed as engagement element 220 applies a pulling force to tub portion 110. A distance of the relative displacing of the tub is equal to at least one thickness of an integrated lancet and test strip. When tub portion 110 is in the relative second position, inserts 185 are not extended as far into wells 199 as when in the relative first position. Also, obstructed openings 805,810 are no longer obstructed by surfaces 815,820—thus forming openings 860,870. With openings 860,870 formed, an advancing mechanism (e.g., pusher and chain system) 550, for example, may enter opening 860 and push next integrated lancet and test strip 190 along surface 195 and out opening 870 for use within testing meter 205. When the example pusher and chain system 550 is retracted, engagement element 220 may return tub portion 110 to its relative first position from cartridge 105, where surfaces 815,820 obstruct openings 860,870 (to form obstructed openings 805,810) again.

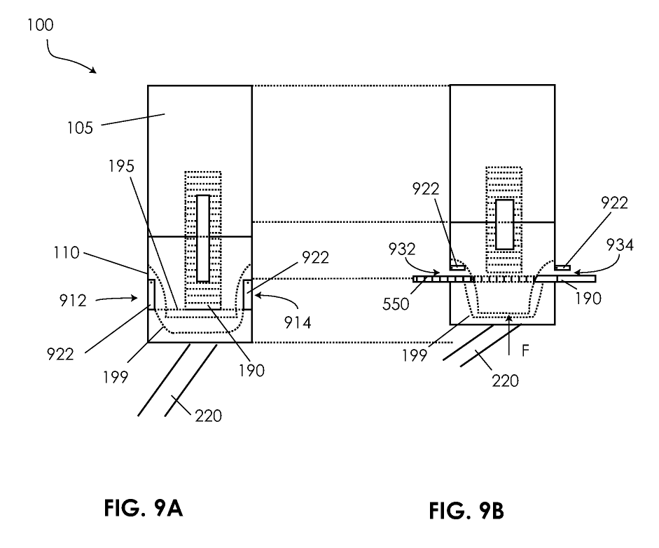

FIGS. 9A-B illustrate openings formed in an ICTA upon application of a force by a testing meter, according to another embodiment. As shown, ICTA 100 comprises cartridge portion 105 coupled to tub portion 110. Tub portion 110 is engaged with engagement element 220 of meter 205. Resilient biasing element 905 is shown coupled to tub portion 110 and to cartridge portion 105. Resilient biasing element 905 is positioned such that the force applied by the resilient biasing elements pushes the cartridge portion 105 and tub portion 110 in opposite directions, away from each other. For instance, resilient biasing element 905 may be under compressive stress and pushing the cartridge portion 105 in the positive y-direction and tub portion 110 in the negative y-direction. ICTA 100 may comprise stops to stop tub portion 110 and cartridge portion 105 when separated a certain distance (so that tub portion 110 cannot be displaced passed a certain stopping point by resilient biasing element 165

FIG. 9A shows tub portion 110 in a relative first position from cartridge portion 105, as maintained by resilient biasing element 905. In the relative first position, tub portion 110 comprises obstructed openings 912,914, which are obstructed by obstruction elements 922 (e.g., flaps). Obstruction elements 922 may provide for a sealed environment for the plurality of integrated lancet and test strips when the tub portion 110 is in the relative first position from the cartridge portion 105. The next integrated lancet and test strip 190 to be advanced is located on surface 195 of tub portion 110. Inserts 185 of cartridge portion 105 is also shown extending into a tub wells 199. In some embodiments, the next integrated lancet and test strip 190 may not be resting on surface 1 95, but rather, still locked in the inserts 185 of cartridge portion 105 and above surface 195, such that inserts 185 are not extending into tub wells 199.

FIG. 9B illustrates tub portion 110 in a relative second position from cartridge portion 105 after engagement element 220 applies a force F to ICTA 100 (in the positive y-direction). Engagement element 220 pushes tub portion 110 into cartridge portion 105 to relatively displace tub portion 110 from cartridge portion 105, in this case relatively displacing tub portion 110 to be closer to cartridge portion 105. Resilient biasing element 905 is stressed (e.g., compressed even further) as engagement element 220 applies a displacing force to tub portion 110. A distance of the relative displacing of the tub is equal to at least one thickness of an integrated lancet and test strip. When tub portion 110 is in the relative second position, inserts 185 are extended further into wells 199 as when in the relative first position. Obstruction elements 922 are flapped open by pusher and chain system 550 to form openings 932,934 as shown in FIG. 9B. Again, the obstruction elements 922 may become unobstructed by the pusher and chain system 550, or alternatively, opened by a mechanism (e.g., spring and hinged door) controlled by the force applied to the ICTA. The pusher and chain system 550 may enter opening 932 and push an integrated lancet and test strip out opening 934 for use within testing meter 205. When the pusher and chain system 550 is retracted, obstruction elements 922 are flapped closed again and engagement element 220 returns tub portion 110 to its relative first position from cartridge 105, where a sealed environment may be provided for test strips 115. In certain alternative embodiments, obstruction elements 922 are not included and openings 932,934 are instead obstructed by a surface of cartridge portion 105. When engagement element 220 applies a displacing force, the surface of the cartridge portion 105 is displaced relative to the obstructed openings, thus allowing the obstructed openings to become unobstructed (similarly as shown in FIGS. 8B).

Operational Sequence of Medical Diagnostic Device

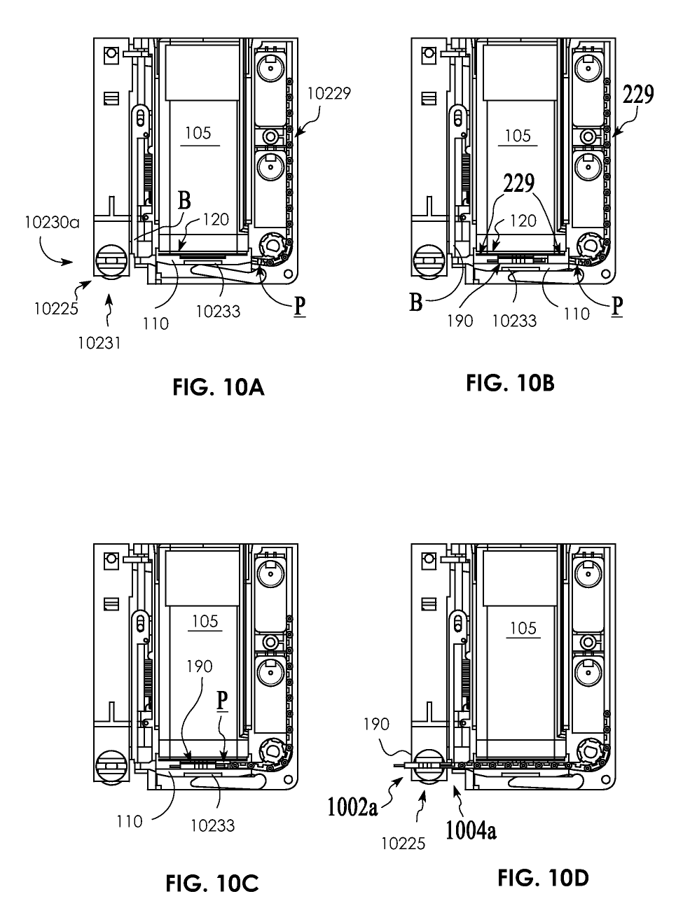

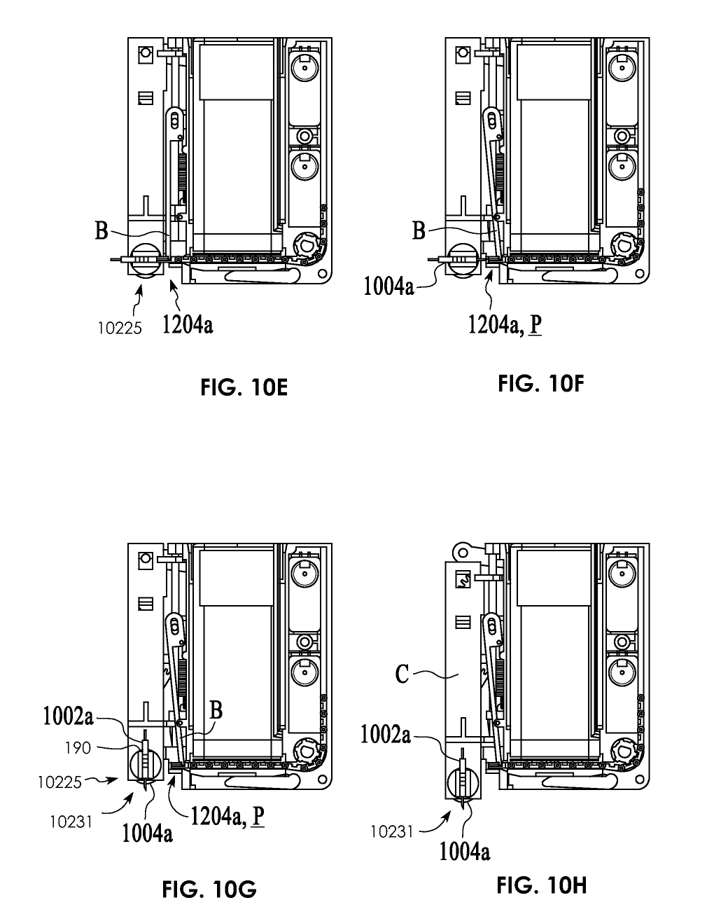

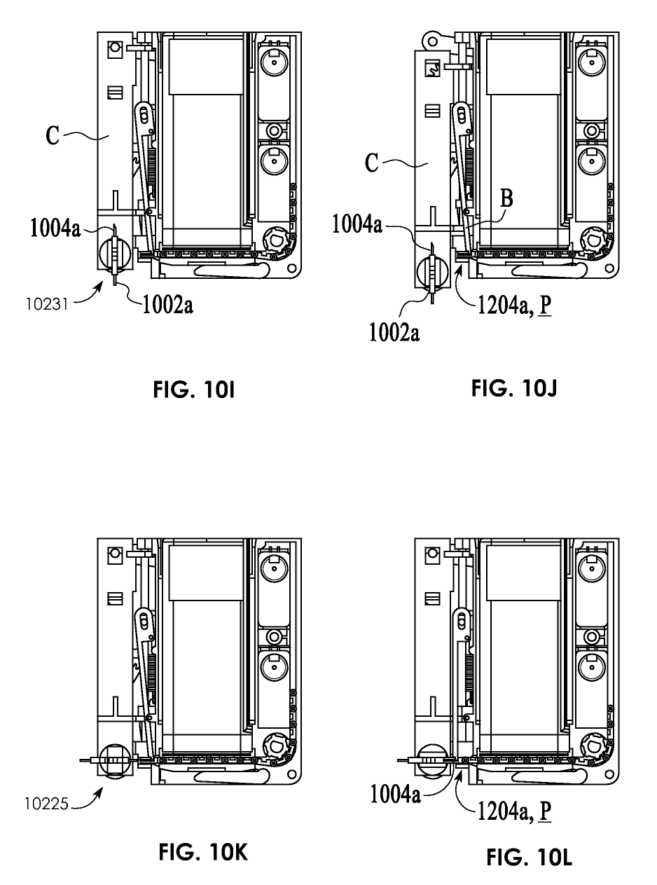

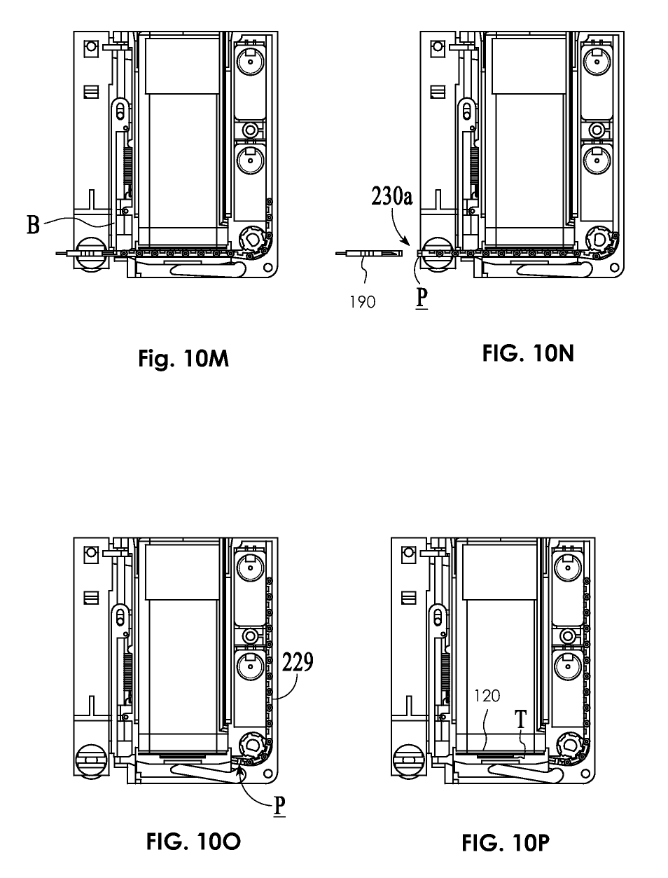

FIGS. 10A-10P illustrate an example operational sequence of a medical diagnostic apparatus, in accordance with certain embodiments. FIG. 10A shows the medical diagnostic apparatus of this embodiment. The turret 10225 is shown with the positions of lancing and testing port 10231 and ejection port 10230a pointed out. The function and operation of the turret 10225, lancing and testing port 10231, and ejection port 10230a are described in sufficient detail for the scope of the underlying principles conveyed herein. A more detailed description and analysis can be found in U.S. patent application Ser. Nos. 11/535,985, 11/535,986, 12/488,181, and 61/102,640 entireties of which is incorporated by reference herein.

A track 229 has a chain therein which is led by pusher P. The cartridge portion 105 has sealing element 120 in between tub portion 110. Sealing element 120 may utilize an o-ring type seal, for example. Furthermore, a surface 195 of the tub portion 110 may include wells 199 in which inserts 185 from the cartridge portion 105 may extend into. Tub portion 110 may include a centering element 10233, which centers a next integrated lancet and test strip for precision loading. In certain alternative embodiments, the integrated lancet and test strip is centered when the inserts of the cartridge portion extend into the wells of the tub portion. A blade B is also illustrated awaiting its time to move downwardly for uncapping a lancet of a next integrated lancet and test strip 190.

FIG. 10B shows the tub portion 110 moved down by a pulling force F applied to the ICTA by engagement element 220, breaking the sealing element 120 with tub portion 110 to expose a next integrated lancet and test strip 190. The next integrated lancet and test strip 190 may be loaded from the cartridge portion 105 onto a track 10229 guided by centering element 10233. The tub portion 110 may include a guide platform for positioning an integrated lancet and test strip while retreating from the cartridge portion 105. The integrated lancet and test strip may therefore be loaded with precision onto the guide track segment from which a pusher P matches a contour of the lancet end of the integrated lancet and test strip and advance the integrated lancet and test strip into a turret 10225. Alternatively, in certain embodiments, inserts 185 of cartridge portion 105 may extend into the wells of the tub portion and center the integrated lancet and test strip as it is advanced along a surface of the tub in between the wells.

FIG. 10C shows the pusher P advanced to meet the next integrated lancet and test strip 190. The tub portion 110 continues to be in the downward position while the track 10229 is exposed. FIG. 10D shows the pusher P after having pushed the next integrated lancet and test strip 190 into turret 10225. The strip end 1002a of the next integrated lancet and test strip 190 is pushed through first, while the lancet end 1004a of the next integrated lancet and test strip 190 is behind. At FIG. 10E, a blade B or decapping lever moves down to engage the lancet cap 1204a. A ridge on the lancet cap 1204a allows a contour of the blade B to couple therewith. The chain retracts as shown in FIG. 10F rotating the blade B slightly to permit the lancet cap 1204a to move rearward along with the chain and pusher P so that the lancet cap 1204a becomes removed from the lancet end 1004a of the next integrated lancet and test strip 190 which remains in position in the turret 10225.

Referring to FIG. 10G, now that the lancet cap 1204a is removed and retracted fully from the next integrated lancet and test strip 190, the turret 10225 is rotated 90 degrees. This 90 degree rotation of the next integrated lancet and test strip 190 orients the next integrated lancet and test strip 190 with lancet 1004a first and strip 1002a behind, for being advanced through port 10231 for lancing.

FIG. 10H illustrates a lancing position as the carriage C is moved relative to the rest of the meter apparatus for lancing. Alternatively, a mechanism for pushing only the integrated lancet and test strip downward or only a turret section of the carriage downward may be provided.

Referring to FIG. 10I, the carriage C is moved back upward after the lancing or piercing of the skin of a diabetic at a lancing site. The turret 10225 is rotated 180 degrees preparing for sensing. Note that the strip end 1002a is shown in FIG. 10I pointing toward port 10231, while in FIGS. 10G and 10H, the lancet end 1004a was pointing toward port 10231.

FIG. 10J illustrates how the carriage C is again moved downward this time for permitting body fluid appearing at the lancing site to be applied to the strip 1002a. Note that the lancet cap 1204a, blade B, and pusher P each remain in position while the lancing and testing occurs. The pusher P is overlapped with the cap 1204a, such that the blade holds both the cap 1204a and pusher P in place.

FIG. 10K shows the carriage C moved back upward, and the turret 10225 having been rotated 90 degrees from when the body fluid was being applied to the strip 1002a. Now at FIG. 10L, the pusher P pushers the cap 1204a back onto the lancet end 1004a.

The next integrated lancet and test strip 190 may protrude from the housing when loaded into the turret 10225. The port 10231 and 10230a may be configured with a slot or may be two ends of a same cavity that curves around the two sides of the housing shown. In this way, the carriage C. advances the next integrated lancet and test strip 190 for lancing and testing, and the turret 10225 may remain translationally fixed relative to the carriage C. The turret 10225 may alternatively move to expose either end of the integrated lancet and test strip 190 through either port. In another embodiment, the carriage C does not move, while the turret 10225 translates to expose the ends of the integrated lancet and test strip 190 in turn through port 10231.

FIG. 10M shows the uncapping lever or blade B moved back up disengaging from the lancet cap 1204a and pusher P. FIG. 10N shows the ejecting of the next integrated lancet and test strip 190. The pusher P is shown after having advanced to push the next integrated lancet and test strip 190 through port 10230a.

At FIG. 10O, the pusher P is retracted back to the start position on the track 10229 that it was in at FIG. 10A. Now the pusher P is out of the way of the tub portion 110, which can move back up as shown at FIG. 10P and meet again with sealing element 120 to protect the integrated lancet and test strips from ambient air, debris, moisture, etc., until a next testing is to be performed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. An integrated cartridge and tub assembly for storing and advancing a plurality of integrated lancet and test strips for use in a testing meter, the assembly comprising:
a cartridge portion for housing a plurality of integrated lancet and test strips; and
a tub portion coupled to the cartridge portion, the tub portion providing a surface for advancing a single integrated lancet and test strip for use in the meter.

2. The integrated cartridge and tub assembly of claim 1, further comprising:
a resilient biasing element coupled to the tub portion and cartridge portion, the resilient biasing element positioning the tub portion in a relative first position from the cartridge portion; and
a sealing element positioned between the cartridge portion and the tub portion, wherein the resilient biasing element provides closing forces to the cartridge portion and tub portion, the closing forces providing an air-tight and moisture-tight environment for the plurality of integrated lancet and test strips.

3. The integrated cartridge and tub assembly of claim 2, wherein when a displacing force is applied to the assembly, the resilient biasing element is stressed and the tub portion is relatively displaced to a relative second position from the cartridge portion, wherein an opening is formed in the assembly when the tub portion is in the relative second position.

4. The integrated cartridge and tub assembly of claim 3, wherein the tub portion is irremovably coupled to the cartridge portion.

5. The integrated cartridge and tub assembly of claim 3, wherein the tub portion is removably coupled to the cartridge portion.

6. The integrated cartridge and tub assembly of claim 3, wherein the opening allows for the advancement of a single integrated lancet and test strip for use in the testing meter.

7. The integrated cartridge and tub assembly of claim 1, further comprising;
a resilient biasing element coupled to the tub portion and cartridge portion, the resilient biasing element positioning the tub portion in a relative first position from the cartridge portion.

8. The integrated cartridge and tub assembly of claim 7, wherein when a displacing force is applied to the assembly, the resilient biasing element is stressed and the tub portion is relatively displaced to a relative second position from the cartridge portion, wherein an opening is formed in the assembly when the tub portion is in the relative second position.

9. The integrated cartridge and tub assembly of claim 8, wherein when the tub portion is in the relative second position, an opening is formed in the assembly.

10. The integrated cartridge and tub assembly of claim 9, wherein the opening is formed between the tub portion and the cartridge portion.

11. The integrated cartridge and tub assembly of claim 8, wherein the assembly comprises an obstructed opening when the tub portion is in the relative first position, the obstructed opening becoming unobstructed when the tub portion is in the relative second position.

12. The integrated cartridge and tub assembly of claim 11, wherein the obstructed opening is in the tub portion of the assembly.

13. The integrated cartridge and tub assembly of claim 11, wherein the obstructed opening is in the cartridge portion of the assembly.

14. The integrated cartridge and tub assembly of claim 11, further comprising:
a sealing element between the cartridge portion and the tub portion, the sealing element providing a sealed environment within the assembly when the opening is obstructed and not when the opening is unobstructed.

15. The integrated cartridge and tub assembly of claim 8, wherein the tub portion interfaces with an engagement element of the testing meter when inserted within the testing meter, the tub portion receiving the displacing force from the engagement element.

16. The integrated cartridge and tub assembly of claim 8, wherein a distance of the relative displacing of the tub portion is equal to at least one thickness of an integrated lancet and test strip.

17. The integrated cartridge and tub assembly of claim 8, wherein the tub portion comprises a surface with one or more wells, and the cartridge portion comprises cartridge inserts aligned with the one or more wells, the cartridge inserts extending further into the wells when the tub portion is in the relative first position than when the tub portion is in the relative second position.

18. The integrated cartridge and tub assembly of claim 1, further comprising:
a sealing element between the cartridge portion and the tub portion, the sealing element to provide a sealed environment when the tub portion is in the relative first position and not when the tub portion is in the relative second position.

19. The integrated cartridge and tub assembly of claim 1, wherein the tub comprises obstruction elements, the obstruction elements providing a sealed environment within the assembly when closed, and providing openings in the assembly for the advancement of a single integrated lancet and test strip when open.

20. The integrated cartridge and tub assembly of claim 19, wherein the obstruction elements are flaps.

21. A disposable integrated cartridge and tub assembly for storing and advancing a plurality of integrated lancet and test strips for use in a testing meter, the assembly comprising:
a cartridge portion for housing a plurality of integrated lancet and test strips;
a tub portion irremovably coupled to the cartridge portion, the tub portion providing a surface for advancing a single integrated lancet and test strip for use in the meter;
a resilient biasing element coupled to the tub portion and cartridge portion, the resilient biasing element positioning the tub portion in a relative first position from the cartridge portion; and
a sealing element positioned between the cartridge portion and the tub portion, wherein the resilient biasing element provides closing forces to the cartridge portion and tub portion, the closing forces providing an air-tight and moisture-tight environment for the plurality of integrated lancet and test strips;
wherein when a displacing force is applied to the assembly, the resilient biasing element is stressed and the tub portion is relatively displaced to a relative second position from the cartridge portion, wherein an opening is formed in the assembly when the tub portion is in the relative second position.

22. A system for analyte monitoring, comprising:
a meter for determining an analyte concentration;
an assembly for storing and advancing a plurality of integrated lancet and test strips for use by the meter during a determination, the assembly for insertion into the meter and comprising:
a cartridge portion for housing a plurality of integrated lancet and test strips; and
a tub portion coupled to the cartridge portion, the tub portion providing a surface for advancing a single integrated lancet and test strip for use in the meter.

23. The system of claim 22, wherein the assembly further comprises:
a resilient biasing element coupled to the tub portion and cartridge portion, the resilient biasing element positioning the tub portion in a relative first position from the cartridge portion; and
a sealing element positioned between the cartridge portion and the tub portion, wherein the resilient biasing element provides closing forces to the cartridge portion and tub portion, the closing forces providing an air-tight and moisture-tight environment for the plurality of integrated lancet and test strips.

24. The system of claim 23, wherein when a displacing force is applied to the assembly, the resilient biasing element is stressed and the tub portion is relatively displaced to a relative second position from the cartridge portion, wherein an opening is formed in the assembly when the tub portion is in the relative second position.

25. The system of claim 24, wherein the tub portion is irremovably coupled to the cartridge portion.

26. The system of claim 24, wherein the tub portion is removably coupled to the cartridge portion.

27. The system of claim 24, wherein the opening allows for the advancement of a single integrated lancet and test strip for use in the testing meter.

28. The system of claim 22, wherein the assembly further comprises;
a resilient biasing element coupled to the tub portion and cartridge portion, the resilient biasing element positioning the tub portion in a relative first position from the cartridge portion.

29. The system of claim 28, wherein when a displacing force is applied to the assembly, the resilient biasing element is stressed and the tub portion is relatively displaced to a relative second position from the cartridge portion, wherein an opening is formed in the assembly when the tub portion is in the relative second position.

30. The system of claim 29, wherein when the tub portion is in the relative second position, an opening is formed in the assembly.

31. The system of claim 30, wherein the opening is formed between the tub portion and the cartridge portion.

32. The system of claim 29, wherein the assembly comprises an obstructed opening when the tub portion is in the relative first position, the obstructed opening becoming unobstructed when the tub portion is in the relative second position.

33. The system of claim 32, wherein the obstructed opening is in the tub portion of the assembly.

34. The system of claim 32, wherein the obstructed opening is in the cartridge portion of the assembly.

35. The system of claim 32, wherein the assembly further comprises:
a sealing element between the cartridge portion and the tub portion, the sealing element providing a sealed environment within the assembly when the opening is obstructed and not when the opening is unobstructed.

36. The system of claim 29, wherein the tub portion interfaces with an engagement element of the testing meter when inserted within the testing meter, the tub portion receiving the displacing force from the engagement element.

37. The system of claim 29, wherein a distance of the relative displacing of the tub portion is equal to at least one thickness of an integrated lancet and test strip.

38. The system of claim 29, wherein the tub portion comprises a surface with one or more wells, and the cartridge portion comprises cartridge inserts aligned with the one or more wells, the cartridge inserts extending further into the wells when the tub portion is in the relative first position than when the tub portion is in the relative second position.

39. The system of claim 29, wherein the assembly further comprises:
a sealing element between the cartridge portion and the tub portion, the sealing element to provide a sealed environment when the tub portion is in the relative first position and not when the tub portion is in the relative second position.

40. The system of claim 22, wherein the tub comprises obstruction elements, the obstruction elements providing a sealed environment within the assembly when closed, and providing openings in the assembly for the advancement of a single integrated lancet and test strip when open.

41. The system of claim 40, wherein the obstruction elements are flaps.

42. A system for analyte monitoring, comprising:
a meter for determining an analyte concentration;
a disposable assembly for storing and advancing a plurality of integrated lancet and test strips for use by the meter during a determination, the disposable assembly for insertion into the meter and comprising:
a cartridge portion for housing a plurality of integrated lancet and test strips;
a tub portion irremovably coupled to the cartridge portion, the tub portion providing a surface for advancing a single integrated lancet and test strip for use in the meter;
a resilient biasing element coupled to the tub portion and cartridge portion, the resilient biasing element positioning the tub portion in a relative first position from the cartridge portion; and
a sealing element positioned between the cartridge portion and the tub portion, wherein the resilient biasing element provides closing forces to the cartridge portion and tub portion, the closing forces providing an air-tight and moisture-tight environment for the plurality of integrated lancet and test strips;
wherein when a displacing force is applied to the assembly, the resilient biasing element is stressed and the tub portion is relatively displaced to a relative second position from the cartridge portion, wherein an opening is formed in the assembly when the tub portion is in the relative second position.
